(19) United States
(12) Patent Application Publication
(10) Pub. No.: US 2011/0106161 A1
(43) Pub. Date: May 5, 2011

(54) Position Retaining Crosslink

(75) Inventors: Bryan S. Wilcox, Collierville, TN (US); John Massey, Hernando, MS (US); Jason Michael May, Cordova, TN (US)

(73) Assignee: WARSAW ORTHOPEDIC, INC., Warsaw, IN (US)

(21) Appl. No.: 12/609,821

(22) Filed: Oct. 30, 2009

(51) Int. Cl. A61B 17/70

(52) U.S. Cl. 606/250

(57) ABSTRACT

A crosslink for a spinal stabilization system is disclosed that provides significant increased torsional stability for the spinal stabilization system. The crosslink includes a first crosslink arm and a second crosslink arm having an eye. An eyebolt is included that has a horizontal passageway for receiving at least a portion of the first crosslink arm and an upper portion for receiving the eye of the second crosslink arm. The eyebolt includes a means for inhibiting rotational and translational movement of the portion of the first crosslink arm.

### BACKGROUND

The present invention concerns spinal fixation systems, and particularly systems utilizing elongated rods adjacent to the spinal column and crosslinks for significantly increasing the torsional stability of the spinal stabilization system. More specifically, the invention concerns improvements to a crosslink that is used to interconnect two approximately parallel elongate members, such as spinal rods, that include a means for inhibiting movement of the arms of the crosslink once positioned in a location desired by the surgeon.

Spinal fixation systems are implanted during a surgical procedure to treat a variety of problems. These treatments include correction of congenital spinal deformations, repair of spinal injuries and fusion of vertebra to stabilize degenerative conditions and alleviate chronic back pain. Several techniques and systems have been developed for correcting and stabilizing the spine and facilitating spinal fusion. In one common system, a longitudinal member, such as a bendable rod, is disposed along the vertebral column and is fixed to various vertebrae along the length of the column by way of a number of fixation elements. Usually, the surgeon first attaches vertebral fixation elements to the spine in appropriate anatomic positions, and then attaches each vertebral fixation element to the spinal rod.

In order to increase the torsional stability of the spinal fixation system, one or more crosslinks may be connected across to each of the rods along the axial plane of the spine. Crosslinks consist of two or more arms that can be locked on a rod by a setscrew. The arms can be adjusted in length and are typically joined by an eyebolt component with a lock nut. Prior to tightening the lock nut and setscrews, the surgeon positions the crosslink assembly in the anatomy. However, current crosslink assemblies do not provide a means to retain the positioning prior to tightening of the assembly, which results in surgeon frustration as the components often move prior to being tightened.

### SUMMARY

According to one aspect a crosslink is disclosed that is configured and operable to inhibit movement of the crosslink components prior to being tightened during the surgical procedure. The crosslink includes a first crosslink arm and a second crosslink arm having an eye. An eyebolt is included having a horizontal passageway for receiving at least a portion of the first crosslink arm and an upper portion for receiving the eye of the second crosslink arm. The eyebolt includes a means for inhibiting rotational and translational movement of the portion of the first crosslink arm. In one form, the means for inhibiting is utilized prior to the first and second crosslink arms being fixedly secured to the eyebolt.

In one form, the means for inhibiting comprises a friction member positioned in a passageway of the eyebolt that extends downwardly and into the horizontal passageway of the eyebolt. In another form, the eyebolt further includes a compressible insert positioned in the passageway and a retainer at least a portion of which is positioned in the passageway. An upper portion of the compressible insert is positioned within a recessed portion of the retainer and a lower portion of the compressible insert is in contact with the friction member. Depression of the retainer causes the compressible insert to compress and exert a force on the friction member that causes the friction member to exert force on the first crosslink arm thereby inhibiting rotational and translational movement of the first crosslink arm. In one representative form, the compressible insert comprises a spring made from biocompatible material.

In yet another form, the means for inhibiting comprises a deformable member positioned in a recessed portion of the horizontal passageway. The deformable member may comprise an O-ring made from a biocompatible material. In another form, the crosslink includes a second means for inhibiting rotational movement of the second crosslink arm prior to the first and second crosslink arms being fixedly secured to the eyebolt. In one form, the second means for inhibiting rotational movement comprises a compressible insert positioned in the eye of the second crosslink arm. The compressible insert preferentially is made from a biocompatible deformable material.

Another aspect discloses a crosslink that is configured and operable to inhibit movement of the crosslink components prior to being tightened during the surgical procedure. The crosslink includes a first and second crosslink arm. The crosslink further includes an eyebolt that has an insert operable to inhibit rotational and translational movement of the first crosslink arm. In one form, the insert is utilized prior to the first crosslink arm and the second cross link arm being fixedly secured to the eyebolt.

A horizontal passage in the eyebolt is included for receiving at least a portion of the first crosslink arm. The horizontal passage includes a recessed portion containing the insert. In this form, the insert comprises an O-ring made from a biocompatible material. In another form, the crosslink further comprises a second insert positioned in the second crosslink arm operable to inhibit rotational movement of the second crosslink arm prior to the first crosslink arm and the second cross link arm being secured to the eyebolt. In one form, the insert comprises a friction member, a compressible insert, and a retainer positioned in a passageway in the eyebolt. A lower surface of the friction member extends into a second passageway of the eyebolt that is configured to receive at least a portion of the first crosslink arm. Upon application of force to the retainer, the retainer compresses the compressible insert thereby applying force to the friction member.

Yet another aspect discloses a crosslink that is configured and operable to inhibit movement of the crosslink components prior to being tightened during the surgical procedure. In this form, the crosslink includes a first crosslink arm and an eyebolt having a first passageway for receiving at least a portion of the first crosslink arm. The first passageway includes a recessed portion that has a compressible member positioned therein and at least a portion of which is exposed in the first passageway. The compressible member is operable to inhibit rotational and translational movement of the first crosslink arm.

Another aspect of this form further comprises a second crosslink arm, wherein the second crosslink arm includes an eye that is configured to be positioned around a portion of the eyebolt. The eye includes a second recessed portion that has a second compressible member positioned therein and at least a portion of which is exposed in a second passageway defined by the eye. The second compressible member is operable to inhibit rotational movement of the second crosslink arm. In another form, the first crosslink arm includes a first end having a counter bore configured to be deformed to prevent removal of the portion of the crosslink arm from the first passageway.

Related features, aspects, embodiments, objects and advantages of the present invention will be apparent from the following description.

### BRIEF DESCRIPTION OF THE DRAWINGS

FIG. 1 is a top elevational view of a spinal fixation construct in accordance with one embodiment of the present invention.

FIG. 2 is an end view of a portion of the construct shown in FIG. 1.

FIG. 3 is a perspective view of a crosslink used in the construct shown in FIG. 1.

FIG. 4 is a perspective view of the crosslink shown in FIG. 3 illustrating a representative means for inhibiting rotational and translational movement of a crosslink arm.

FIG. 5 is a cross-sectional view of a portion of the crosslink illustrated in FIG. 3.

FIG. 6 is a cross-sectional view of a portion of another representative crosslink illustrated in FIG. 3.

FIG. 7 is a cross-sectional view of a portion of another representative crosslink illustrated in FIG. 3.

FIG. 8 is a cross-sectional view of a portion of another representative crosslink.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

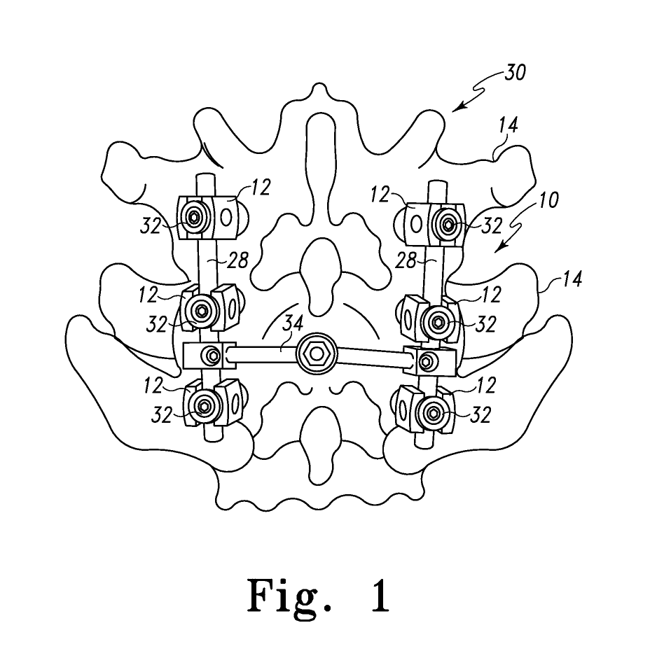

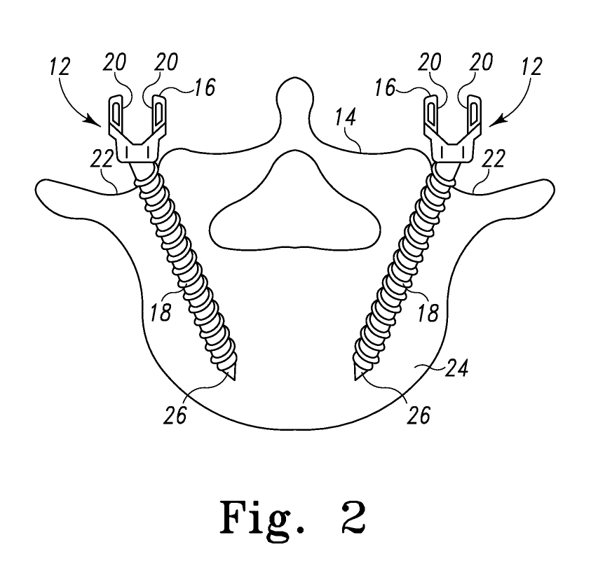

Referring to FIG. 1, a construct or spinal stabilization system 10 is illustrated that is utilized to treat a variety of spinal conditions. In this form, construct 10 includes a plurality of multi-axial screws 12 that are inserted into select portions of vertebrae 14 to be instrumented. As illustrated in FIG. 2, in this representative form multi-axial screws 12 include a screw head 16 and a threaded shaft 18 extending downwardly from the screw head 16. As illustrated, screw head 16 comprises a U-shaped member or cradle that includes two opposing internally threaded sidewalls 20. In one form, multi-axial screws 12 are preferentially made from titanium and comprise top-loading screws. In this form, the threaded shaft 18 of the multi-axial screws 12 is inserted through a pedicle 22 of the vertebrae 14 and into a body 24 of the vertebrae 14. In one form, multi-axial screws 12 have a self-tapping flute 26 that obviates the need for tapping. However, in cases of dense, sclerotic, or osteoporotic bone, tapping is often recommended prior to insertion of the multi-axial screw 12.

Referring back to FIG. 1, after insertion of the multi-axial screws 12 in the desired locations, a rod 28 is inserted into the U-shaped screw heads 16 such that the rod 28 extends along a sagittal plane of the spine 30. As illustrated, in this example rod 28 is positioned in three U-shaped screw heads 16 but other numbers of multi-axial screws 12 may be used in other surgical procedures. The rods 28 preferentially run substantially parallel to one another along the sagittal plane of the spine 30. Once the rods 28 are in proper position, set screws or plugs 32 are screwed into the screw heads 16 thereby securing the rods 28 to the multi-axial screws 12. To provide further stabilization of the spine 30, a crosslink 34 is connected to each respective rod 28 that runs substantially perpendicular to the rods 28. As such, in this form the crosslink 34 runs along an axial plane of the spine 30. Although only one crosslink 34 is illustrated in FIG. 1, one or more crosslinks 34 may be used in other surgical procedures. As with the multi-axial screws 12, the rods 28 and crosslinks 34 may be manufactured from titanium or any other biocompatible material that is strong enough to provide the stabilization desired to be obtained from the surgical procedure.

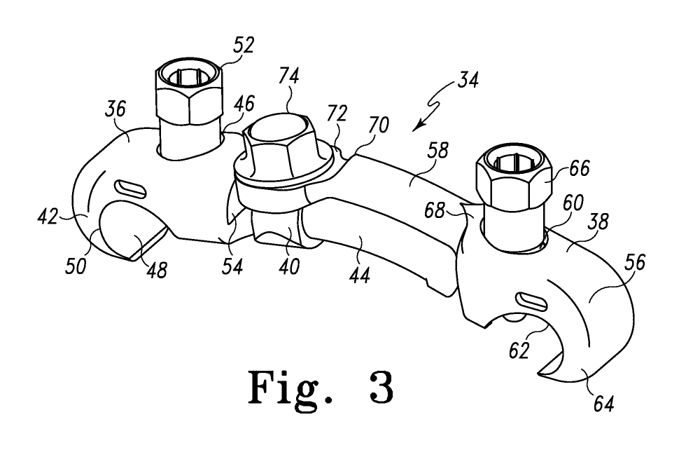

Referring to FIG. 3, the crosslink 34 is used to significantly increase the torsional stability of the construct 10. In this form, crosslink 34 comprises a first crosslink arm 36, a second crosslink arm 38, and an eyebolt 40. The first crosslink arm 36 includes a hook segment 42 and a rod or extension segment 44. The hook segment 42 includes a threaded aperture 46 that extends vertically or downwardly through the hook segment 42 and into a portion of a hook 48. In this form, hook 48 comprises a passageway through an end 50 of the hook segment 42. Referring collectively to FIGS. 1 and 3, the hook 48 is configured and sized to receive one of the rods 28. Once the rod 28 is positioned in the hook 48, a setscrew 52 is positioned or screwed into the threaded aperture 46 and tightened thereby securing the first crosslink arm 36 to the rod 28. In one form, the setscrew 52 is configured and sized to have a recessed fit in the threaded aperture 46. The rod segment 44 of the first crosslink arm 36 extends horizontally away from a second end 54 of the hook segment 42. The rod segment 44 has a generally circular cross-sectional configuration in this representative example, but may have other cross-sectional configurations (e.g.—rectangular, square, octagonal, and so forth) in other representative forms.

The second crosslink arm 38 includes a hook segment 56 and an arm or extension segment 58. As with the first crosslink arm 36, the hook segment 56 includes a threaded aperture 60 that extends vertically or downwardly through the hook segment 56 and into a portion of a hook 62. In this form, hook 62 comprises a passageway through an end 64 of the hook segment 56. Referring collectively to FIGS. 1 and 3, the hook 62 is configured and sized to receive one of the rods 28. Once the rod 28 is positioned in the hook 62, a setscrew 66 is positioned or screwed into the threaded aperture 60 and tightened thereby securing the second crosslink arm 38 to the rod 28. In one form, the setscrew 66 is configured and sized to have a recessed fit in the threaded aperture 60. Referring back to FIG. 3, the arm segment 58 of the second crosslink arm 38 extends horizontally away from a second end 68 of the hook segment 56. A distal end 70 of the arm segment 58 includes an eye 72 that, as set forth in greater detail below, is used to secure the second crosslink arm 38 to the eyebolt 40. In particular, a nut 74 is used to secure the second crosslink arm 38 to the eyebolt 40. In this representative form, the arm segment 58 has a generally circular cross-sectional configuration, but may have other cross-sectional configurations (e.g.—rectangular, square, octagonal, oval and so forth) in other representative forms.

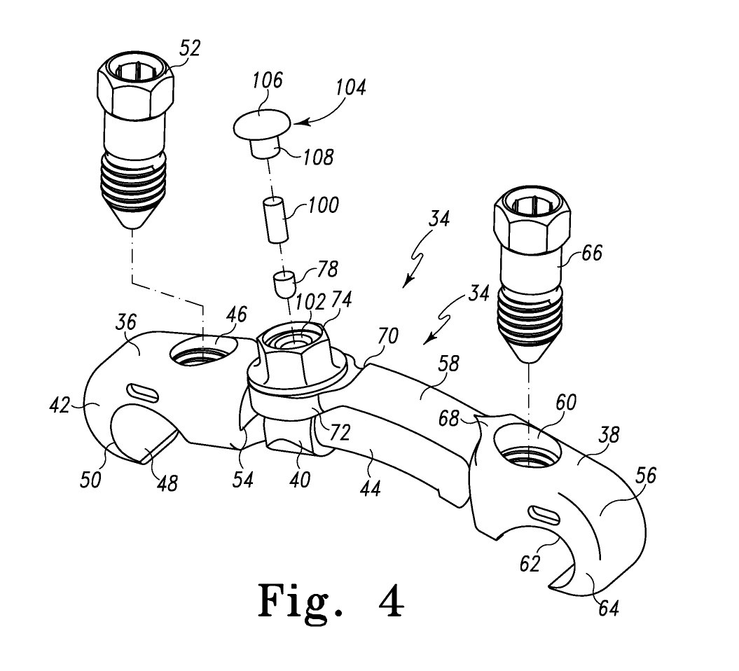

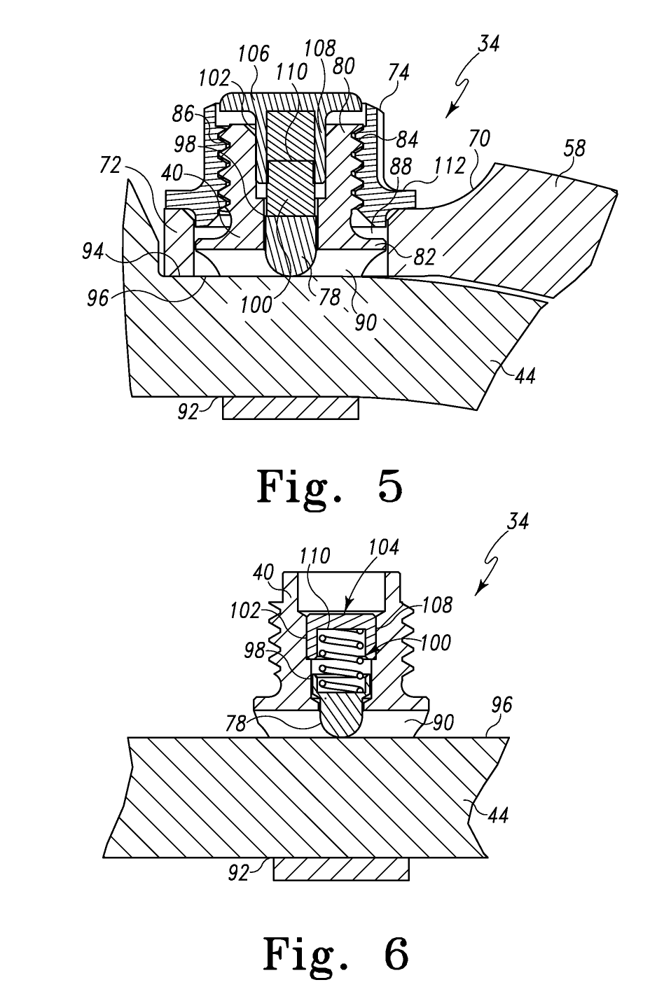

Referring collectively to FIGS. 4 and 5, in this representative form the crosslink 34 includes a friction member 78 that is positioned inside the eyebolt 40 that is operable to inhibit movement of the rod segment 44 of the first crosslink arm 36 once positioned in eyebolt 40. Prior to tightening the lock nut 74 and setscrews 52, 66, the surgeon positions the crosslink 34 in the anatomy of the patient. The friction member 78 provides a means for the surgeon to maintain the position of the rod segment 44 in the eyebolt 40 prior to the crosslink 34 being tightened in position. In particular, friction member 78 is operable to prevent rotational and translational (i.e.—horizontal) movement of the rod segment 44 prior to the nut 74 being tightened on the eyebolt 40.

As illustrated best in FIG. 5, the eyebolt 40 includes a post or upper portion 80 that extends upwardly from a central portion 82 of the eyebolt 40. The post 80 includes an externally threaded portion 84 that is configured and sized to threadably engage an internally threaded portion 86 of the nut 74. The eye 72 of the second crosslink arm 38 includes a vertical aperture or passageway 88 configured and sized to receive the central portion 82 of the eyebolt 40. A lower portion 90 of the eyebolt 40 includes a horizontal aperture or passageway 92 that is configured and sized to receive the rod segment 44 of the first crosslink arm 36. As such, during assembly the rod segment 44 of the first crosslink arm 36 is slid through the horizontal passageway 92 of the eyebolt 40 to a desired position. Then, the arm segment 58 of the second crosslink arm 38 is positioned over the top of the post 80 where it is permitted to travel down the post 80 until reaching the central portion 82 of the eyebolt 40. At this point, a lower portion 94 of the arm segment 58 makes contact with an upper portion 96 of the rod segment 44 of the first crosslink arm 36 thereby preventing the arm segment 58 from traveling any further down the eyebolt 40.

As further illustrated in FIGS. 4 and 5, the post 80 of the eyebolt 40 includes a first vertical aperture or passageway 98 configured and sized to receive at least a portion of the friction member 78. As illustrated, a lower portion of the friction member 78 extends through the vertical passageway 98 of the eyebolt 40 and into the horizontal passageway 92 in the lower portion 90 of the eyebolt 40. A compressible insert 100 is positioned in a portion of the vertical passageway 98 and extends upwardly into a second larger aperture or passageway 102 of the eyebolt 40. A retainer 104 is included that has a cap 106 and an insert 108 that includes a recessed portion 110 sized and configured to receive an upper portion of the compressible insert 100. As illustrated, the outside diameter of the insert 108 is sized and configured to provide a friction fit with the second passageway 102 of the eyebolt 40. The inside diameter of the recessed portion 110 is sized and configured to receive the upper portion of the compressible insert 100.

During the surgical procedure, once the eyebolt 40 is positioned on the rod segment 44 of the first crosslink arm 36 in the desired position, the surgeon can press down on the cap 106 of the retainer 104 which in turn causes the compressible insert 100 to compress and exert a downward force on the friction member 78. The downward force on the friction member 78 causes the friction member 78 to engage an upper surface of the rod segment 44 of the first crosslink arm 36 thereby providing provisional retention of the eyebolt 40 on the rod segment 44 of the first crosslink arm 36. As such, both rotational and translational movement of the first crosslink arm 36 in relation to the eyebolt 40 is inhibited so that the surgeon can place the second crosslink arm 38 on the eyebolt 40 without having to worry about the eyebolt 40 moving from the desired position on the rod segment 44 of the first crosslink arm 36. This eliminates the frustration that surgeons experience by parts moving during the surgical procedure. In an alternative forms, the present invention may be pre-assembled during manufacture and the friction member 78 may be depressed downwardly by the retainer 104 during manufacture.

The second crosslink arm 38 can then be placed in proper position by placing the aperture 88 of the eye 72 over the post 80 until select portions of the arm segment 58 and the eye 72 make contact with the upper surface or portion 96 of the rod segment 44 of the first crosslink arm 36. At this point, the nut 74 can be threaded on the post 80 of the eyebolt 40 to secure the first and second crosslink arms 36, 38 to one another. In yet another form, the second crosslink arm 38 can be positioned over the post 80 and in contact with the upper surface 96 of the rod segment 44 of the first crosslink arm 36 prior to compression of the retainer 104. The nut 74 can then be threaded onto the post 80 and prior to tightening of the nut 74, the retainer 104 can be depressed thereby causing the friction member 78 to engage the rod segment 44 of the first crosslink arm 36. Again, depression of the retainer 104 causes the compressible insert 100 to compress thereby exerting a downward force on the friction member 78. In this form, the compressible insert 100 comprises a deformable elastomer, but other types of deformable biocompatible materials can be used as well.

Referring to FIG. 6, in yet another form of the present invention the compressible insert 100 comprises a spring preferentially made from a biocompatible material such as titanium. An upper portion of the spring 100 is positioned in the recessed portion 110 of the retainer 104. A lower portion of the spring 100 is in contact with an upper portion of the friction member 78. As with the other form, as the retainer 104 is pressed downwardly into the second passageway 102 of the eyebolt 40, the retainer 104 compresses the spring 100 thereby causing a force to be exerted on the rod segment 44 of the first crosslink arm 36 by the friction member 78. This force inhibits rotational and translational movement of the first crosslink arm 36 thereby making the crosslink 34 easier to install during the surgical procedure. In addition, this force inhibits movement of the eyebolt 40 during assembly of the construct 10.

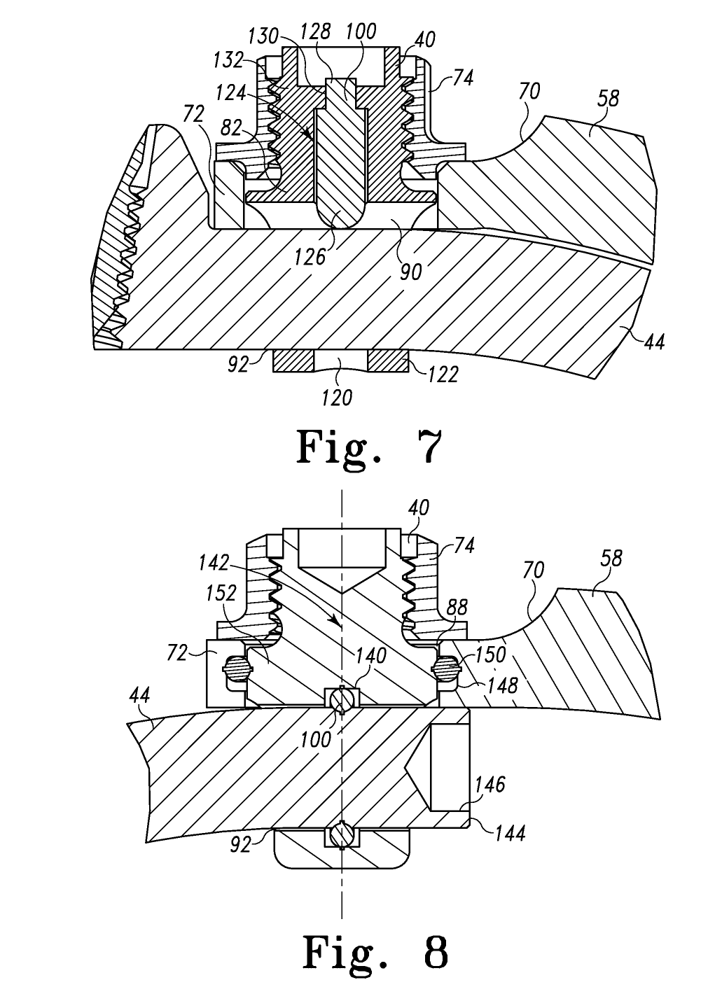

Referring to FIG. 7, in another form of the present invention the compressible insert 100 is inserted into the eyebolt 40 through a hole 120 in a bottom surface 122 of the eyebolt 40. In this form, the eyebolt 40 includes a first bore 124 aligned with the hole 120 that is sized and configured to receive the compressible insert 100. The first bore 124 is preferentially sized such that the compressible insert 100 is friction fit into the first bore 124 so that it will not readily fall out of the first bore 124. A lower portion 126 of the compressible insert 100 extends downwardly from the first bore 124 and into a portion of the passageway 92 such that it makes contact with the upper surface 96 of the rod segment 44 of the first crosslink arm 36. In this form, the compressible insert 100 inhibits rotational and translational movement of the first crosslink arm 36. An upper portion 128 of the compressible insert 100 extends through a first passageway 130 in the eyebolt 40. The first passageway 130 has a diameter smaller than that of the first bore 124. As illustrated, the compressible insert 100 extends from the central portion 90 of the eyebolt 40 toward an upper portion 132 of the eyebolt 40 and is located about a central axis of the eyebolt 40.

Referring to FIG. 8, in yet another form of the present invention the horizontal passageway 92 of the eyebolt 40 includes a recessed portion 140 that is sized and configured to receive a compressible insert 100. In the illustrated form, the recessed portion 140 comprises a circular-shaped recess in the passageway 92 that runs along a central vertical axis 142 of the eyebolt 40. The compressible insert 100 can be a biocompatible elastomer molded into the shape of an O-ring. Once the rod segment 44 of the first crosslink arm 36 is inserted into the horizontal passageway 92, the compressible insert 100 inhibits rotational and translational movement of the first crosslink arm 36. In addition, in another representative form an end 144 of the rod segment 44 is provided with a counter bore 146 that allows the end 144 of the rod segment 44 to be deformed during installation. When the end 144 of the rod segment 44 is deformed, this prevents the rod segment 44 from disengaging the eyebolt 40 by being removed from the horizontal passageway 92 in the eyebolt 40. The end 144 of the rod segment 44 is deformed in an asymmetric manner as to not interfere with translation and rotation between the first crosslink arm 36 and the second crosslink arm 38.

As further illustrated in FIG. 8, in yet another form of the present invention the vertical aperture 88 of the eye 72 of the second crosslink arm 38 includes a second recessed portion 148 that is sized and configured to receive a second compressible insert 150. The second recessed portion 148 runs horizontally within the vertical aperture 88 of the eye 72. In this form, the second compressible insert 150 makes contact with a central portion 152 of the eyebolt 40 when the second crosslink arm 38 is positioned on the eyebolt 40. The second compressible insert 150 inhibits free rotation of the second crosslink arm 38 about the central portion 152 of the eyebolt 40. In addition, the second compressible insert 150 inhibits vertical movement of the second crosslink arm 38 prior to the nut 74 being threaded onto the eyebolt 40.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A crosslink, comprising:
a first crosslink arm;
a second crosslink arm having an eye; and
an eyebolt having a horizontal passageway for receiving at least a portion of said first crosslink arm and an upper portion for receiving said eye of said second crosslink arm, wherein said eyebolt includes a means for inhibiting rotational and translational movement of said first crosslink arm.

2. The crosslink of claim 1, further comprising a second means for inhibiting rotational movement of said second crosslink arm.

3. The crosslink of claim 2, wherein said second means for inhibiting comprises a compressible insert positioned in said eye of said second crosslink arm.

4. The crosslink of claim 3, wherein said compressible insert comprises a biocompatible deformable material.

5. The crosslink of claim 1, wherein said means for inhibiting comprises a friction member positioned in a passageway of said eyebolt that extends downwardly and into said horizontal passageway.

6. The crosslink of claim 5, wherein said eyebolt further includes a compressible insert positioned in said passageway and a retainer at least a portion of which is positioned in said passageway, wherein an upper portion of said compressible insert is positioned within a recessed portion of said retainer and a lower portion of said compressible insert is in contact with said friction member.

7. The crosslink of claim 6, wherein depression of said retainer causes said compressible insert to compress and exert a force on said friction member thereby causing said friction member to exert force on said first crosslink arm thereby inhibiting rotational and translational movement of said first crosslink arm.

8. The crosslink of claim 6, wherein said compressible insert comprises a spring.

9. The crosslink of claim 1, wherein said means for inhibiting comprises a deformable member positioned in a recessed portion of said horizontal passageway.

10. The crosslink of claim 9, wherein said deformable member comprises an O-ring.

11. A crosslink, comprising:
a first crosslink arm;
a second crosslink arm; and
an eyebolt including an insert operable to inhibit rotational and translational movement of said first crosslink arm.

12. The crosslink of claim 11, further comprising a horizontal passage in said eyebolt for receiving at least a portion of said first crosslink arm, wherein said horizontal passage includes a recessed portion containing said insert.

13. The crosslink of claim 12, wherein said insert comprises an O-ring.

14. The crosslink of claim 11, further comprising a second insert positioned in said second crosslink arm operable to inhibit rotational movement of said second crosslink arm.

15. The crosslink of claim 11, wherein said insert comprises a friction member, a compressible insert, and a retainer positioned in a passageway in said eyebolt, wherein a lower surface of said friction member extends into a second passageway of said eyebolt configured to receive at least a portion of said first crosslink arm.

16. The crosslink of claim 15, wherein upon application of force to said retainer said retainer compresses said compressible insert thereby applying force to said friction member.

17. The crosslink of claim 15, wherein said compressible insert comprises a spring.

18. A crosslink, comprising:
a first crosslink arm; and
an eyebolt having a first passageway for receiving at least a portion of said first crosslink arm, wherein said first passageway includes a recessed portion that has a compressible member positioned therein and at least a portion of which is exposed in said first passageway, wherein said compressible member is operable to inhibit rotational and translational movement of said first crosslink arm.

19. The crosslink of claim 18, further comprising a second crosslink arm, wherein said second crosslink arm includes an eye configured to be positioned around a portion of said eyebolt, wherein said eye includes a second recessed portion that has a second compressible member positioned therein and at least a portion of which is exposed in a second passageway defined by said eye, wherein said second compressible member is operable to inhibit rotational movement of said second crosslink arm.

20. The crosslink of claim 18, wherein said first crosslink arm includes a first end having a counter bore configured to be deformed to prevent removal of said portion of said crosslink arm from said first passageway.
